(19) United States
(12) Patent Application Publication
(10) Pub. No.: US 2018/0282271 A1
(43) Pub. Date: Oct. 4, 2018

(54) ANTIBIOTIC AMINO ACID MIMETICS

(71) Applicant: BIOXINESS PHARMACEUTICALS, INC., Hercules, CA (US)

(72) Inventors: Mansour BASSIRI, Hercules, CA (US); Richard D. Gless, Hercules, CA (US)

(21) Appl. No.: 15/936,198

(22) Filed: Mar. 26, 2018

Related U.S. Application Data
(60) Provisional application No. 62/476,015, filed on Mar. 24, 2017.

(51) Int. Cl. C07C 323/60; C07D 209/20; A61P 31/04

(52) U.S. Cl. CPC C07C 323/60; A61P 31/04; C07D 209/20

(57) ABSTRACT

Disclosed are methionine mimetic compounds that possess antibiotic properties in prokaryotic cells. The compounds of this invention comprise a dipeptide having a methionine mimetic bound to an aromatic amino acid. Such compounds exhibit significantly improved antibacterial properties.

### FIELD OF THE INVENTION

This invention is directed to methionine mimetics that possess antibiotic properties in prokaryotic cells. These mimetics are coupled to an amino acid selected from, for example, tryptophan, phenylalanine and tyrosine. When so coupled, these dimeric peptides are capable of utilizing well known prokaryotic transport mechanisms so as to be internalized by these cells. Upon internalization, intracellular peptidases convert the dimeric peptides into their corresponding amino acids, The methionine mimetics described herein then binds with specificity to the tRNA^{Met} synthesase. This blocks the addition of methionine to initiation as well as elongation steps in the growing peptide chain ultimately resulting in cell death. Accordingly, disclosed are compounds, compositions and methods for treating a prokaryotic infection in a mammal as well as prodrugs for such compounds.

### STATE OF THE ART

Prokaryotic cells such as bacteria import amino acids by specific transporters that include those that transport dimeric peptides. These transporters have been utilized in U.S. Pat. No. 8,580,859 to incorporate antibiotic methionine mimetics into bacteria. As disclosed, such mimetics are coupled to generic classes of natural and unnatural amino acids optionally substituted with a large number of substituents to provide for dimeric peptides. Once taken up/ transported into bacteria, the methionine mimetic prevents proper peptide synthesis by these bacteria resulting in bacterial death.

These compounds provided efficacious results as measured by in vitro assays indicating acceptable levels of intrabacterial methionine mimetics. However, significantly greater antibacterial properties for such dipeptides would be advantageous as such would lead to greater and more rapid bacterial death.

### SUMMARY OF THE INVENTION

This invention is based, in part, on the discovery that certain specific L-amino acids provide significantly improved dipeptide uptake by prokaryotic cells when attached to methionine mimetics. Once internalized, these dipeptides are converted to their corresponding single amino acid components by, for example, enzymatic processes. The methionine mimetic then binds with high specificity to the tRNA^{Met} synthesase thereby inhibiting protein synthesis in the bacteria, which leads to bacterial growth inhibition and cell death.

In one of its compound aspects, this invention is directed to a compound of formula I:

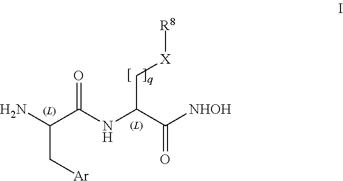

where:
R⁸ is selected from the group consisting of hydrogen and C₁-C₄ alkyl;
Ar is selected from the group consisting of phenyl, 4-hydroxyphenyl and 3-indolyl;
q is 1, 2, 3, or 4;
X is selected from the group consisting of S, O, SO, SO₂ and CH₂; and
each (L) indicates an L isomer at that stereochemical center;
including pharmaceutically acceptable salts and/or solvates thereof. In another embodiment, Ar is selected from the group consisting of phenyl, phenyl glycine, 4-hydroxyphenyl, and 3-indolyl. In another embodiment, formula I can include a histidine group instead of the Ar group.

In one embodiment, X is S, R⁸ is methyl, q is 1 or 2 and Ar is phenyl.

In one embodiment, X is S, R⁸ is methyl, q is 1 or 2 and Ar is 4-hydroxyphenyl.

In one embodiment, X is S, R⁸ is methyl, q is 1 or 2 and Ar is 3-indolyl.

Specific compounds within the scope of this invention include the following L, L isomers:

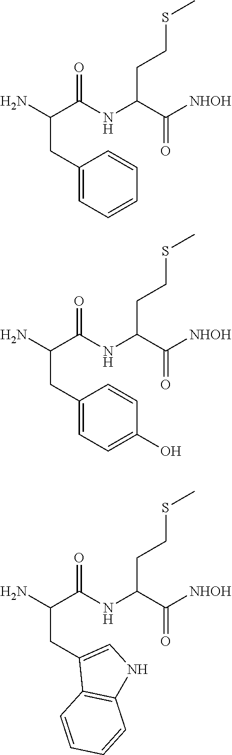

In one embodiment, this invention provides for a pharmaceutical composition comprising a pharmaceutically acceptable carrier and a compound of formula I.

This invention also provides for bacteria comprising within it intracellular space a mixture of a compound of formula I above as well as the L isomer of a compound of formula II:

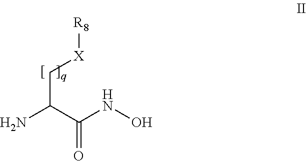

where X, q and R⁸ are as defined above; and including salts and/or hydrates thereof.

In one of its method aspects, this invention is directed to a method for killing prokaryotic cells which method comprises administering to said cells a compound of formula I above.

In one embodiment, the prokaryotic cells are bacterial cells.

In one embodiment, the bacterial cells are E. coli bacteria.

In one embodiment, this invention provides for a method of treating a subject with a bacterial infection which method comprises administering to the subject an effective amount of a compound of formula I or a pharmaceutical composition comprising an effective amount of a compound of formula I.

### BRIEF DESCRIPTION OF THE DRAWINGS

FIGS. 1A and 1B demonstrate the superior antibacterial properties of an aromatic amino acid, phenylalanine, attached to a methionine mimetic as compared to glycine attached to the same methionine mimetic. FIGS. 2-4 show additional data.

### DETAILED DESCRIPTION OF THE INVENTION

This invention provides for compounds and methods for killing prokaryotic cells and, in particular, pathogenic bacterial cells. However, prior to addressing this invention in more detail, the following terms will be defined.

### 1. Definitions

As used herein, the following definitions shall apply unless otherwise indicated. Further, if any term or symbol used herein is not defined as set forth below, it shall have its ordinary meaning in the art.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of this invention. Procedures for inserting such labels into the compounds of this invention will be readily apparent to those skilled in the art based on the disclosure herein.

“Alkyl” refers to monovalent saturated aliphatic hydrocarbyl groups having from 1 to 4 carbon atoms and preferably 1 to 2 carbon atoms. This term includes, by way of example, linear and branched alkyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Cₓ alkyl refers to an alkyl group having x number of carbon atoms.

The compounds of this invention may exist as solvates, especially hydrates. Hydrates may form during manufacture of the compounds or compositions comprising the compounds, or hydrates may form over time due to the hygroscopic nature of the compounds. Compounds of this invention may exist as organic solvates as well, including DMF, ether, and alcohol solvates among others. The identification and preparation of any particular solvate is within the skill of the ordinary artisan of synthetic organic or medicinal chemistry.

“Subject” refers to a mammal. The mammal can be a human or non-human animal mammalian organism.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Treating” or “treatment” of a disease or disorder in a subject refers to 1) preventing the disease or disorder from occurring in a subject that is predisposed or does not yet display symptoms of the disease or disorder; 2) inhibiting the disease or disorder or arresting its development; or 3) ameliorating or causing regression of the disease or disorder.

“Pharmaceutically acceptable” refers to a material that is not biologically or otherwise undesirable, e.g., the material may be incorporated into a pharmaceutical formulation administered to a subject without causing and significant undesirable biological effects or interfering in a deleterious manner with any of the other components of the formulation in which it is contained.

“Pharmaceutically acceptable carrier” refers to materials such as solvents, stabilizers, pH-modifiers, tonicity modifiers, adjuvants, binders, diluents and other materials well known to the skilled artisan that are suitable for administration to a subject in combination with the compound or compounds of this invention. The specific carrier selected is predicated in part on the intended route of administration such as rectal, oral, intravenous, parenteral, topical, inhalation, and the like. Such is well within purview of the skilled artisan.

An “effective amount” refers to that amount that results in a desired pharmacological or physiological effect for a specific condition such as an infection. In some cases, an effective amount is that amount sufficient to treat the symptoms of the disease or condition. In some cases, an effective amount is that amount sufficient to partially or completely cure the subject of the disease or condition. In reference to bacterial infections, an effective amount is preferably that amount that reduces the number of bacterial cells, inhibit bacterial growth, and/or kill existing bacteria. In some cases, an effective amount is that amount that is provided to a subject to prevent a bacterial infection when the subject is at risk of such an infection.

### 2. Compounds of the Invention

The compounds of this invention are directed to compounds of formula I:

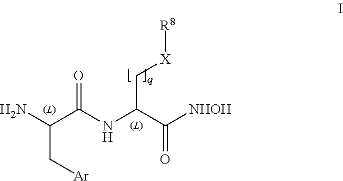

where:
R¹ is selected from the group consisting of hydrogen and C₁-C₄ alkyl;
Ar is selected from the group consisting of phenyl, 4-hydroxyphenyl and 3-indolyl; q is 1, 2, 3, or 4;
X is selected from the group consisting of S, O, SO, SO₂ and CH₂; and
each (L) indicates an L isomer at that stereochemical center;
including pharmaceutically acceptable salts and/or solvates thereof.

In one embodiment, X is S, R⁸ is methyl, q is 1 or 2 and Ar is phenyl.

In one embodiment, X is S, R⁸ is methyl, q is 1 or 2 and Ar is 4-hydroxyphenyl.

In one embodiment, X is S, R⁸ is methyl, q is 1 or 2 and Ar is 3-indolyl.

Specific compounds within the scope of this invention include the following L, L isomers:

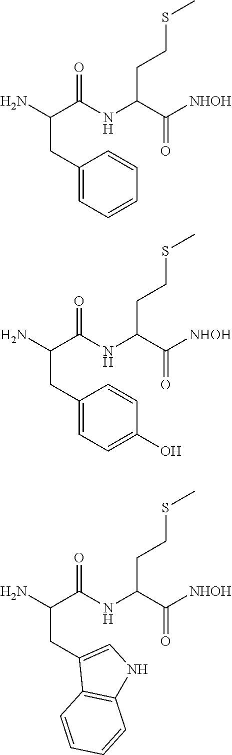

In each case, one or more of the compounds described herein can be formulated into a pharmaceutical composition comprising a pharmaceutically acceptable carrier and an effective amount of such compound or compounds.

### 3. Synthesis

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, N.Y., 1999, and references cited therein.

If the compounds of this invention contain one or more chiral centers, such compounds can be prepared or isolated as pure stereoisomers, i.e., as individual enantiomers or d(1) stereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Sigma-Aldrich (Milwaukee, Wis., USA), Bachem (Torrance, Calif., USA), Emka-Chemce or others). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

### Synthesis of Representative Compounds of the Invention

In one general embodiment, the compounds comprise a methionine mimetic coupled to an aromatic amino acid. The methionine mimetics of formula I are readily prepared from the N-protected methyl ester of methionine as shown below:

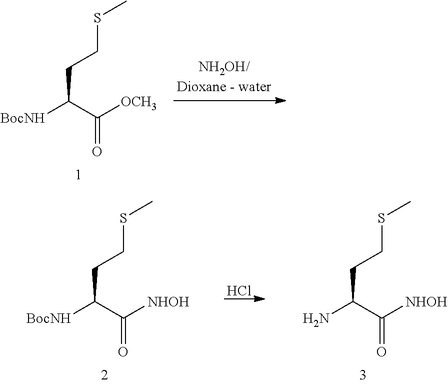

Specifically, the N-Boc protected methyl ester of methionine (1) is treated with hydroxylamine in a solvent mixture of dioxane and water so as to provide for compound (2). That compound can be isolated or purified by conventional conditions such as chromatography, precipitation, crystallization and the like or, alternatively, used in the next step without isolation and/or purification. Subsequently, the Boc protecting group is removed by conventional conditions such as the addition of an acid such as HCl so as to provide for compound (3). Again, that compound can be isolated or purified by conventional conditions such as chromatography, precipitation, crystallization and the like.

Compound (3) is then coupled to an aromatic amino acid (4) using conventional amino acid coupling conditions well known in the art as shown in the following reaction scheme:

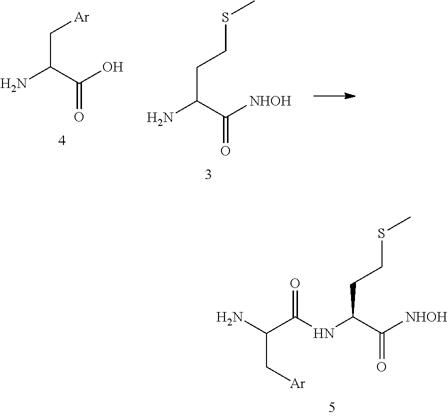

where Ar is the aromatic portion of amino acid (4). Histidine can be also used having an imidazole group. Upon coupling completion, the resulting compound is isolated and purified as described above.

For compounds where q is 1 in compound (3), the reaction can start with cysteine and proceeds as above for R⁸ hydrogen groups. For R⁸ alkyl groups, alkylation of the —SH group of an otherwise suitably protected cysteine compound proceeds via conventional alkylation techniques well known in the art.

For compounds where X is SO or SO₂, such compounds correspond to the sulfoxide and sulfones and are readily prepared by oxidizing the sulfur with a mild oxidizing agent such as metachloroperbenzoic acid using conventional techniques.

For compounds where X is CH₂, such compounds are readily prepared by starting with L-2-amino-n-hexanoic acid [CH₃(CH₂)₃CH(NH₂)COOH] and following the procedures set forth above.

### 4. Formulations and Methods of Use

In general, the compounds and compositions of this invention are useful in killing prokaryotic cells such as bacteria. As such, these compounds and compositions are capable of treating bacterial infections in subjects when administered thereto in an effective amount. Examples of bacteria and bacterial infections that are treatable by the compounds and compositions described herein include, without limitation, Escherichia coli, Pseudomonas aeruginosa, Klebsiella pneumonia, Acinetobacter baumannii, Neisseria gonorrhoeae, Haemophilus influenza, Clostridium difficile (C. diff), Enterobacter faecalis, Staphylococcus aureus, Methicillin Resistant Staphylococcus aureus (MRSA), Serratia marcescens, Helicobacter pylori, Saccharomyces cervisiae, Streptococcus thermophiles, Lactococcus lactis, Streptococcus agalactiae, Beta Hemolytic streptococcus, Mycobacterium bovis, Listeria monocytogenes, Peptostreptococcus micros, Fusobacterium nucleaturm, Porphyromonas gingivalis, Salmonella tyrphimurium, and/or Bacciluss subtillus, which may infect, for example, wounds, skin, eyes, ears, nose and/or the GI tract.

In some embodiments, the compounds and compositions of this invention are capable of inhibiting bacterial growth and, accordingly, are useful as bactericidal, antibacterial, and anti-infective agents.

In some embodiments, the compounds and compositions of this invention are capable of inhibiting intracellular bacterial protein synthesis by at least 20%, or by at least 50%, or by at least 75%, or by at least 90%, or by at least 95% or 100% when compared to intracellular bacterial protein synthesis in the absence of the compounds and compositions described herein.

In some embodiments, the compounds and compositions of this invention are capable of intrabacterial inhibition of methionyl-tRNA synthetase by at least 20%, or by at least 50%, or by at least 75%, or by at least 90%, or by at least 95% or 100% when compared to the enzymatic activity in the absence of the compounds and compositions described herein.

In some embodiments, the compounds of this invention are effective when administered to a subject in a therapeutically effective amount. Preferably such amounts range from about 0.1 μg/kg to about 300 mg/kg when administered orally, intravenously, intra-arterially, intraperitoneally, intramuscularly, subcutaneously, intraocularly, rectally, transdermally, intrapulmonarily, and the like. In some embodiments, the amounts so administered more preferably range from about 1 μg/kg to about 40 mg/kg.

In some embodiments, the compounds and compositions are administered topically such as cream, ointment, lotion, and the like. When so applied, the amount of compound employed in such topical formulations ranges from 0.1 mg/mL to about 100 mg/mL.

In all cases, the amount of compound administered to the subject depends upon the weight, age, sex, severity of the condition to be treated and other factors well known to the skilled clinician. In some embodiments, the compounds of this invention can be administered at least once a day, preferably once or twice a day, and in some cases, three or more times a day.

### FORMULATION EXAMPLES

The following are representative pharmaceutical formulations containing a compounds of this invention.

### Formulation Example 1—Tablet Formulation

The following ingredients are mixed intimately and pressed into single scored tablets.

| | Quantity per |
|---|---|
| Ingredient | tablet, mg |
| compound of this invention | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| compound of this invention | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this invention with Witepsol® H-15 (triglycerides of saturated vegetable fatty acid; Riches-Nelson, Inc., New York), and has the following composition:

| Ingredient | Amount |
|---|---|
| Compound of this invention | 500 mg |
| Witepsol ® H-15 | Balance |

The following synthetic and biological examples are offered to illustrate this invention and are not to be construed in any way as limiting the scope of this invention. Unless otherwise stated, all temperatures are in degrees Celsius.

### 5. Examples

This invention is further understood by reference to the following examples, which are intended to be purely exemplary of this invention. This invention is not limited in scope by the exemplified embodiments, which are intended as illustrations of single aspects of this invention only. Any methods that are functionally equivalent are within the scope of this invention. Various modifications of this invention in addition to those described herein will become apparent to those skilled in the art from the foregoing description and accompanying figures. Such modifications fall within the scope of the appended claims.

In the examples below, the following abbreviations have the following meanings. If an abbreviation is not defined, it has its generally accepted meaning.
calcd=calculated
g=gram
M+H=molecular mass plus proton
mL=milliliter
mmol=millimol
MS=Mass Spectroscopy
N=Normal
μg=microgram

### A. Chemistry

Example 1: Synthesis of (2)-2-amino-N-hydroxy-4-(methylthio)butanamide hydrochloride (HCl salt, S-enantioner of I-AA)

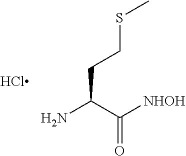

To a solution of (S)-methyl 2-amino-4-(methylthio)butanoate (5 g, 30.7 mmol) in dioxane (50 mL) and water (20 mL) at room temperature was added sodium carbonate (5.3 g, 50 mmol) and Boc anhydride (7.96 g, 36.8 mmol). The mixture was stirred overnight at room temperature followed by dioxane removal under vacuum. The aqueous layer was extracted with ethyl acetate (3x). The combined organic layers were washed with 1N HCl, brine, and dried over anhydrous sodium sulfate, filtered and concentrated to give a residue. That residue was purified on a silica gel column to give (S)-methyl 2-(2-tert-butoxycarbonylamino)-4-(methylthio)butanoate (5.1 g, 63%). MS calcd for (C₁₁H₂₁NO₄S+H)⁺:264.1; MS found: (M+H)⁺=264.1, 164.1 (minus the t-Boc group).

A solution of (S)-methyl-2-(tert-butoxycarbonylamino)-4-(methylthio)butanoate (1 g, 3.8 mmol) in dioxane (10 mL)and hydroxylamine (50% in water, 10 mL) was stirred at room temperature for 2 days. The solution was diluted with ethyl acetate (200 mL). The organic layer was washed with 1 N HCl, brine, and dried over anhydrous sodium sulfate, filtered and concentrated to give a residue, which was purified on a silica gel column (hexane:ethyl acetate, 1:1 to pure ethyl acetate) to give (S)-tert-butyl 1-(hydroxyamino)-4-(methylthio)-1-oxobutan-2-ylcarbamate (0.33 g, 33%). MS calcd for (C₁₀H₂₀N₂O₄S+H)+: 265.1; MS found: (M+H)+=266.2, 166.2 (minus the t-Boc group).

To solid (S)-tert-butyl 1-(hydroxyamimo)-4-(methylthio)-1-oxobutan-2-ylcarbamate (0.33 g, 1.25 mmol) was added 4 N HCl in dioxane (2 mL, 8 mmol). The mixture was stirred at room temperature for one hour, and concentrated. The residue was titrated with ether, and dried to provide the title compound (S)-2-amino-N-hydroxy-4-(methylthio)butanamide hydrochloride (0.18 g, 80%). MS calcd for (C₅H₁₂N₂O₂S−H)⁺:163.1; MS found: (M−H)⁺=163.0; ¹NMR (MeOH-d)δ: 3.82, m, 1H; 2.55, m, 2H; 2.12, s, 3H; 1; 2.1, m, 2H.

Example 2: Synthesis of L-Tryptophan-(S)-2-amino-N-hydroxy-4-(methylthio)butanamide hydrochloride (HCl salt, S-enantioner)

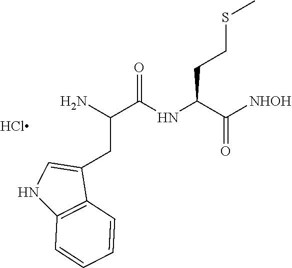

To a solution of (S)-methyl 2-amino-4-(methylthio)butanoate and approximately 1.2 equivalents of Boc-tryptophan in dichloromethane at room temperature was added approximately 1.2 equivalents of diisopropylcarbodiimide and approximately 1.2 equivalents of diisopropylethylamine. The mixture was stirred at room temperature until the reaction was substantially complete, washed with 1 N HCl, brine and dried over sodium sulfate, filtered and concentrated. The title compound was recovered by silica gel chromatography. ¹NMR (MeOH-d)δ: 9.72, m, 1H; 7.68, m, 1H; 7.40, m, 1H; 7.12, m, 4 H; 4.44, m, 1H; 4.18, m, 1H; 3.4, m, 1H; 3.18, m, 1H; 2.52, m, 2H; 2.1, s, 3H; 2.0, m, 2H;

Example 3: Synthesis of L-phenylalanine-(S)-2-amino-N-hydroxy-4-(methylthio)butanamide hydrochloride (HCl salt, S-enantioner)

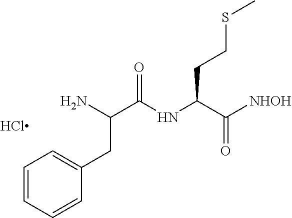

To a solution of (S)-methyl 2-amino-4-(methylthio)butanoate and approximately 1.2 equivalents of Boc-phenylalanine in dichloromethane at room temperature was added approximately 1.2 equivalents of diisopropylcarbodiimide and approximately 1.2 equivalents of diisopropylethylamine. The mixture was stirred at room temperature until the reaction was substantially complete, washed with IN HC1, brine and dried over sodium sulfate, filtered and concentrated. The title compound was recovered by silica gel chromatography. ¹NMR (MeOH-d)δ: 7.32, bm, 5H; 4.44, m, 1H; 4.15, m, 1H; 3.25, m, 1H; 3.0, m, 1H; 2.52, m, 2H; 2.1, s, 3H; 2.0, m, 2H;

Example 4: Synthesis of L-tyrosine-(S)-2-amino-N-hydroxy-4-(methylthio)butanamide hydrochloride (HCl salt, S-enantioner)

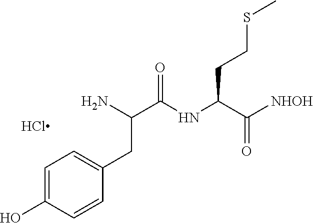

To a solution of (S)-methyl 2-amino-4-(methylthio)butanoate and approximately 1.2 equivalents of Boc-tyrosine in dichloromethane at room temperature was added approximately 1.2 equivalents of diisopropylcarbodiimide and approximately 1.2 equivalents of diisopropylethylamine. The mixture was stirred at room temperature until the reaction was substantially complete, washed with 1N HCl, brine and dried over sodium sulfate, filtered and concentrated. The title compound was recovered by silica gel chromatography. ¹NMR (MeOH-d)δ: 7.1, d, 2H; 6.89, d, 2H; 4.43, m, 1H; 4.07, m, 1H; 3.19, m, 1H; 2.92, m, 1H; 2.52, m, 2H; 2.1, s, 3H; 2.0, m, 2H;

### B. Biology

### Comparative Compounds

The following L,L-dipeptides were tested for their minimum inhibitory concentrations against three different bacterial strains as set forth in the table below. The testing protocol followed conventional methods and identified a MIC value reported as micrograms per milliliter (μg/mL). Each of the dipeptides had the following structure:

TABLE 1

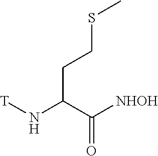

| T | Compound | E. coli | S. aur. | P. aer. |
|---|---|---|---|---|
| Gly | A | 138 | 84 | 647 |
| Ala | B | 158 | 131 | 1040 |
| Val | C | 520 | 416 | 833 |
| Pro | D | 312 | 125 | 270 |
| Lys | E | 312 | 104 | >1660 |
| Glu | F | 554 | 312 | 1660 |

The above results illustrate that of the compounds tested, compound A was most active against E. coli and S. aureus.

### Example 5—Side Chain Substitution of the Methionine Mimetic.

Based on the above results, the sidechain of the methionine mimetic was modified to evaluate the effect of such substitution on MIC values. Again, this test used conventional assays to measure the MIC values of each of these modified mimetics. The results of this test are set forth in Table 2 below:.

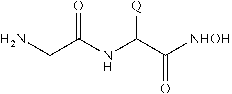

| Q = | Compound | E. coli | S. aur. | P. aer. |
|---|---|---|---|---|
| —CH₂CH₂SCH₃ | A | 138 | 84 | 647 |
| —CH₂CH₂SOCH₃ | G | 123 | 51 | 416 |
| —CH₂CH₂SO₂CH₃ | H | 208 | 104 | 416 |
| —CH₂CH₂CH₂CH₃ | I | 69 | 51 | 520 |
| —CH₂CH₂SCH₂CH₃ | J | 129 | 60 | 554 |
| —CH₂CH₂S(CH₂)₃CH₃ | K | 150 | 60 | 138 |
| —CH₂CH₂SC(CH₃)₃ | L | 207 | 77 | 624 |
| —CH₂SCH₂CH₃ | M | 159 | 104 | 832 |
| —CH₂SC(CH₃)₃ | N | 195 | 77 | 693 |
| —CH₂CH₂OCH₃ | O | 374 | 112 | 624 |

The date in Table 2 demonstrates that there is flexibility in the side-chain of the methionine mimetic including q equal to 1 or 2, X=S, O, SO, SO₂ and CH₂.

### Example 6—Zone of Inhibition Test

Compound A was tested against L-phenylalanine-(S)-2-amino-N-hydroxy-4-(methylthio)butanamide hydrochloride (Example 3) in a side-by-side comparison in a conventional zone of inhibition test. In this test, both compounds were compared at various concentrations against E. coli (ATTC 8739) grown in a Petri dish using the antibiotic kanamycin as a control.

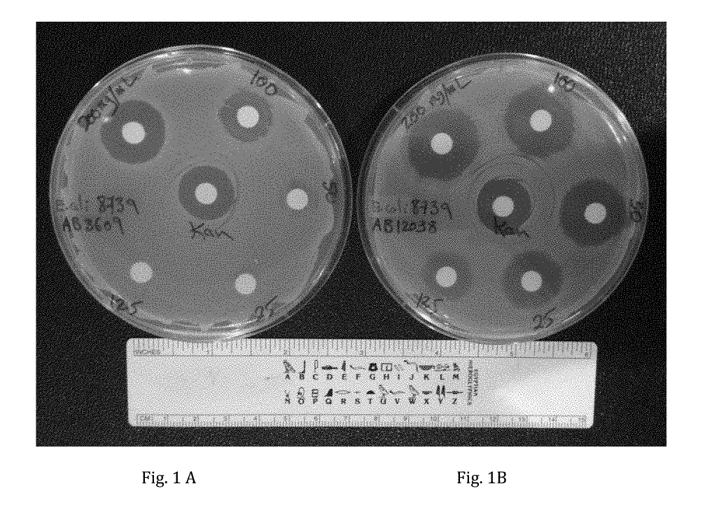

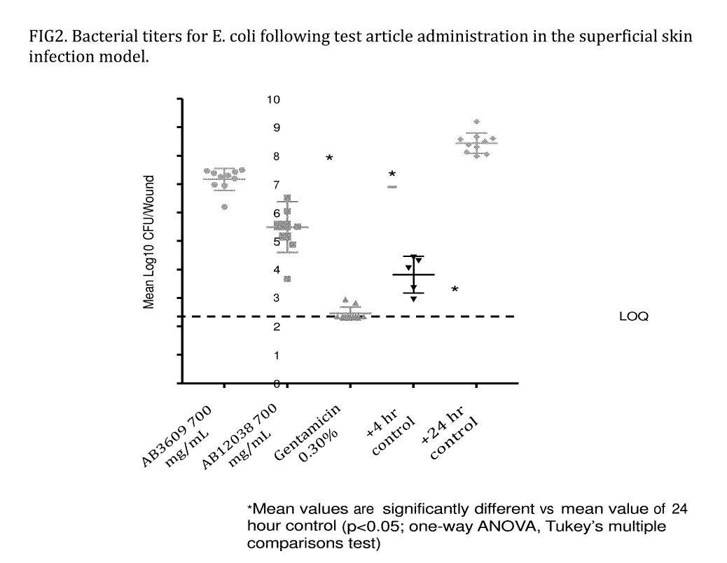

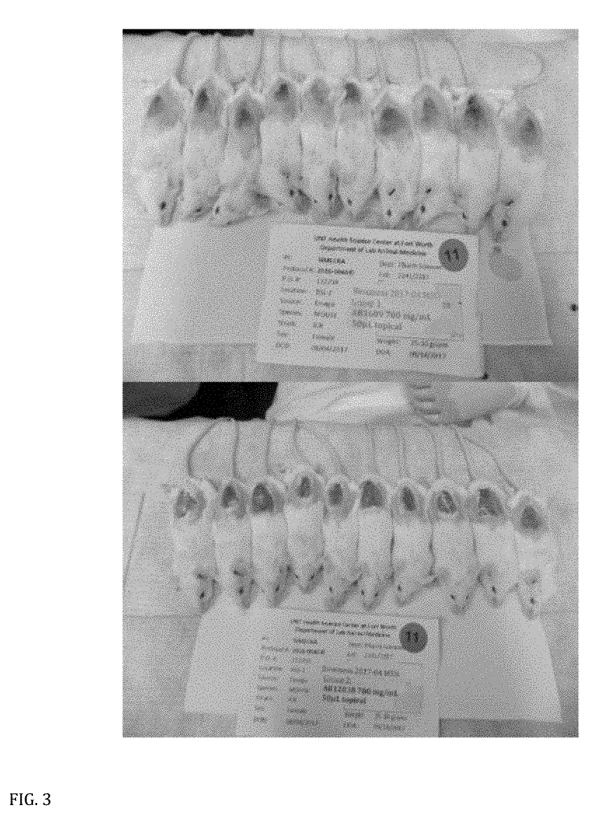

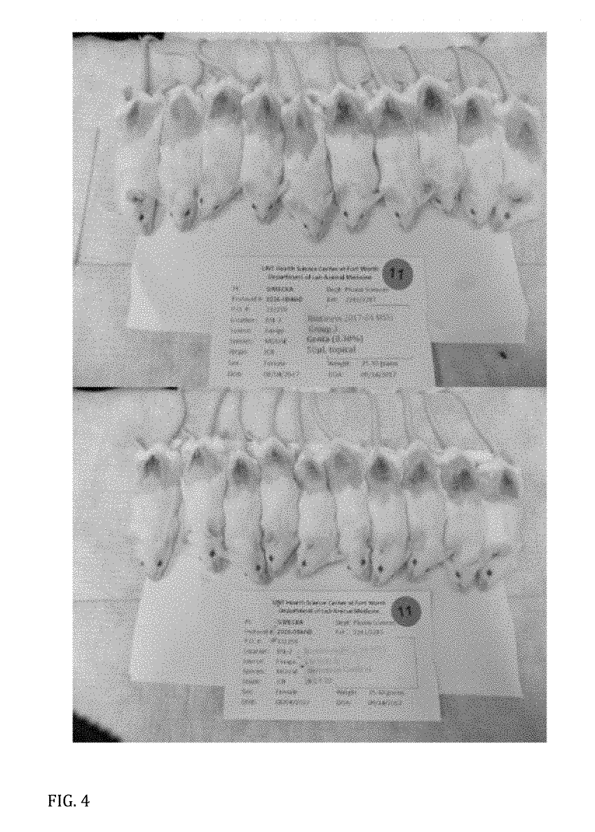

The results are illustrated in FIG. 1A (cmp. A) and FIG. 1B (Ex. 3). As is very clear, cpd. A did not show any noticeable zone of inhibition until the concentration of that compound reached 50 μg and even there, the zone was quite weak.

In contrast thereto, Ex. 3 demonstrated a zone of inhibition at a concentration of 12.5μg wherein that zone was substantially stronger than the zone of inhibition for cpd. A at 50 μg.

Taken together, this data demonstrates that phenylalanine, and by extension other aromatic amino acids, are significantly more active in killing bacterial cells than compound A when combined with the methionine mimetic. Still further, as the aromatic amino acids facilitate transport across the prokaryotic cell wall and are not involved in inhibiting tRNA^{Met} synthesase, the improved efficacy evidenced by FIG. 1A and FIG. 1B must correlate to the improved intracellular concentration of the methionine mimetic. Still further, the data provided in Table 2 above showing efficacy of other methionine mimetic compounds reasonably correlates to a conclusion that such compounds also will exhibit improved efficacy.

While some embodiments have been illustrated and described, a person with ordinary skill in the art, after reading the foregoing specification, can effect changes, substitutions of equivalents and other types of alterations to the compounds of this invention or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

This invention is also not to be limited in terms of the particular aspects described herein, which are intended as single illustrations of individual aspects of this invention. Many modifications and variations of this invention can be made without departing from its spirit and scope, as will be apparent to those skilled in the art. Functionally equivalent methods within the scope of this invention, in addition to those enumerated herein, will be apparent to those skilled in the art from the foregoing descriptions. Such modifications and variations are intended to fall within the scope of the appended claims. It is to be understood that this invention is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of this invention indicated only by the appended claims, definitions therein and any equivalents thereof.

The embodiments, illustratively described herein, may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially” of will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of this invention. This includes the generic description of this invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

### ADDITIONAL EMBODIMENTS AND WORKING EXAMPLES I

### An Evaluation of the In Vitro Activity of Investigational Methionyl-tRNA Synthetase Inhibitors Against a Broad Spectrum of Pathogens

### Introduction

We developed molecules that target methionyl-tRNA synthetase (MetRS) of bacteria for use as antimicrobials. One measure of the antibacterial activity of an agent is by determining the lowest concentration able to inhibit bacterial growth in vitro, or its minimum inhibitory concentration (MIC). The MIC is determined using dilution methods in accordance with guidelines established by the Clinical and Laboratory Standards Institute (CLSI; 1-5).

It had previously been determined that testing in CLSI standard medium such as cation-adjusted Mueller-Hinton broth or other rich media inhibit the activity of the investigational compounds, presumably from binding to media components. For this reason, alternative media must be used to evaluate the in vitro activity of these agents.

In this study, the susceptibility of an array of Gram-positive and Gram-negative bacteria to 5 investigational MetRS inhibitors was evaluated by broth microdilution in accordance with CLSI guidelines (1-5), with the exception that M9 minimal medium was used as the growth medium. In addition, the MIC was determined concurrently using M9 supplemented with 1% and 5% of CLSI standard media.

### MATERIALS AND METHODS

### Test Agents

Five compounds were shipped from Bioxiness Pharmaceuticals to Micromyx and stored at −20° C. Stocks were made in dH₂O at 10,240 μg/mL fresh on each day of the assay. Comparator drugs were supplied by Micromyx and were handled as described below:

| | | | | Stock |
|---|---|---|---|---|
| Comparator | | | Solvent/ | Concentration |
| Drug | Supplier | Lot No. | Diluent | (μg/mL) |
| Ceftazidime | USP | I1K237 | dH₂O | 2,560 |
| Ciprofloxacin | USP | J1L040 | dH₂O | 160 |
| Meropenem | USP | J0K434 | dH₂O | 160 |
| Mupirocin | USP | G0M003 | dH₂O | 10,240 |

### Test Organisms

The test organisms were originally received from clinical sources or from the American Type Culture Collection (ATCC, Manassas, Va.). When received, the organisms were subcultured onto an appropriate agar medium. Following incubation, colonies were harvested from the plate and a cell suspension was prepared in broth containing cryoprotectant and frozen at −80° C. Prior to the assay, a frozen vial of the culture was partially thawed and the contents were streaked for isolation onto an appropriate growth medium. The agar plates were incubated overnight at 35° C. Test organisms included staphylococci and enterococci (Table 1), streptococci (Table 2), Enterobacteriaceae (Table 3), lactose non-fermenting Gram-negative bacilli (Table 4), Haemophilus influenzae (Table 5), Neisseria gonorrhoeae (Table 5), and Clostridium difficile (Table 6). ATCC quality control organisms were included during testing and were evaluated with standard CLSI media as shown in Table 7.

### Test Media

The M9 minimal medium employed for the broth dilution MIC assay consisted of M9 minimal salts (Becton Dickinson, Sparks, Md.; Lot No. 0187624), glucose (Sigma, St. Louis, Mo.; Lot No. BCBL1023V), MgSO₄ (Macron, Center City, Pa.; Lot No. K21606), and CaCl₂ (Macron; Lot No. 9687). M9 was also supplemented with 1% and 5% of the CLSI recommended media for broth MIC testing (CLSI; 1-5), which included Brucella broth (Becton Dickinson, Sparks, Md.; Lot No. 6155858) for anaerobes, Haemophilus Test Media (Remel; Lot No. 895120) for Haemophilus spp., and cation-adjusted Mueller Hinton Broth (MHB II-BD; Lot No. 6117994) for the remaining aerobes. For N. gonorrhoeae, a modified medium described by the ATCC capable of supporting the growth of N gonorrhoeae was used. This media contained Oxoid Special Peptone (Oxoid, Hampshire, UK; Lot No. 1280296), corn starch (Ward's Science; Rochester, N.Y.; Lot No. AD-13344-14), NaCl (Sigma; Lot No. SLBL0434V), K₂HPO₄ (Sigma; Lot No. 052K0147), and KH₂PO₄ (Sigma; Lot No. SLBD5759V).

Supplements for all fastidious isolates and anaerobes were added at full strength to the M9 medium as follows: laked horse blood (Cleveland Scientific, Lot No. 322799) at 3% for streptococci and 5% for anaerobes; vitamin K (Sigma; Lot No. 108K1088) at 1 μg/mL and hemin (Sigma; Lot No. SLBC4685V) at 5 μg/mL for anaerobes; Iso VitaleX (BD; Lot No. 5246843) at 1% for gonococci.

The standard CLSI broth medium was employed for the purposes of quality control of the comparator drugs for the MIC assay and was only used for testing of relevant ATCC quality control isolates as established by CLSI (1-5).

### MIC Assay Procedure

MIC values were determined using a broth microdilution method as recommended by CLSI (1-5). Automated liquid handlers (Multidrop 384, Labsystems, Helsinki, Finland; Biomek 2000 and Biomek F/X, Beckman Coulter, Fullerton Calif.) were used to conduct serial dilutions and make liquid transfers.

The wells of standard 96-well microdilution plates (Costar 3795) were filled with 150 μL of sterile dH₂O in columns 2-12 using the Multidrop 384. Column 1 was filled with 300 μL of the investigational and control agents at the appropriate stock concentration (40× of the highest test concentration). The Biomek 2000 was used to make eleven 2-fold serial dilutions to create a “mother plate.” The wells of column 12 contained no drug and served as growth control wells.

The “daughter plates” were loaded with 185 μL of the media described above using the Multidrop 384. The daughter plates were prepared on the Biomek FX instrument which transferred 5 μL of drug solution from each well of the mother plate to the corresponding well of each daughter plate in a single step.

Standardized inoculum of each organism was prepared per CLSI methods (1-5). The inoculum for each organism was dispensed into sterile reservoirs divided by length (Beckman Coulter), and the Biomek 2000 was used to inoculate the plates. Daughter plates were placed on the Biomek 2000 work surface reversed so that inoculation took place from low to high drug concentration. The Biomek 2000 delivered 10 PL of standardized inoculum into each well resulting in a target inoculum size of approximately 5×10⁵ CFU/mL. The wells of the daughter plates ultimately contained 185 μL of media, 5 μL of drug solution at 40× the test concentration, and 10 μL of bacterial inoculum prepared in media.

Plates were stacked 3-4 high, covered with a lid on the top plate, placed into plastic bags, and incubated at 35° C. under the appropriate conditions, with C. difficile incubated anaerobically. Plates were viewed post-incubation at 18-24 hr and again at 48 hr from the bottom using a plate viewer. An un-inoculated solubility control plate was observed for evidence of drug precipitation. MICs were read where visible growth of the organism was inhibited.

### RESULTS AND DISCUSSION

The susceptibility testing data for the evaluated isolates is presented by organism and medium type in Tables 1-6. MIC values for ATCC quality control organisms as determined with standard CLSI media were within the established QC ranges for the evaluated comparators as shown in Table 7. There was no evidence of drug precipitation for the evaluated agents in any test media with the exception of AB3609 where there was cloudy precipitation from 64-256 μg/mL in media containing 3% lysed horse blood.

The results will be summarized by organism group below.

### Staphylococci and Enterococci (Table 1)

The activity of the MetRS test agents and comparators was evaluated against 4 S. aureus (1 wild type isolate, 1 MRSA, and 2 mupirocin-resistant isolates), 2 S. epidermidis (1 wild type isolate and 1 MRSE), and 2 E. faecalis (1 wild type isolate and 1 VRE). Based on the activity observed in M9 alone against staphylococci, test compound AB3609 was the most active followed by AB12039 and AB12040. AB12038 was active but with higher MICs and AB3354 was largely inactive. The observed activity of the test compounds was greatly diminished upon supplementation of M9 with 1% and 5% of the standard CLSI medium with the exception of AB3609 which maintained similar activity against staphylococci after supplementation with 1% standard CLSI medium. E. faecalis grew poorly in M9 alone, and therefore evaluation of activity was limited to M9 supplemented with 1% and 5% standard CLSI medium where growth was sufficient to determine inhibitory endpoints. AB3609 appeared to be the most active test compound followed by AB12038, AB12039, and AB12040 which all had similar activity. AB3354 was the least active of the test compounds. Upon supplementation of M9 with 5% CLSI standard medium, activity was not apparent at 48 hr for any of the test agents. Activity with the comparator agents was largely as expected based on the phenotype of the selected isolates and, with a few exceptions (e.g. mupirocin-resistant S. aureus for mupirocin and MRSA for meropenem and cefepime), activity was not drastically affected in M9 supplemented with 1% or 5% CLSI standard medium relative to M9 alone.

### Streptococci (Table 2)

The activity of the MetRS test agents and comparators was evaluated against 2 S. pneumoniae (1 wild type isolate, 1 penicillin- and erythromycin-resistant isolate) and 2 S. pyogenes (1 wild type isolate and 1 erythromycin-resistant isolate).

In general, streptococci grew very poorly or not at all in M9 medium alone. In instances where growth in M9 alone was sufficient for reading, little activity was observed for the test compounds with the exception of S. pneumoniae MMX 5354 where comparable activity was observed with AB12038, AB12039, AB12040, and AB3354; AB3609 had no apparent activity. Upon supplementation of M9 with 1% or 5% CLSI standard medium, in instances where sufficient growth was available for interpretation, no activity was observed with the test compounds.

Activity with the comparator agents was largely as expected based on the phenotype of the selected isolates and, with a few exceptions (e.g. penicillin-resistant S. pneumoniae for cefepime and mupirocin), activity was not drastically affected in M9 supplemented with 1% or 5% CLSI standard medium relative to M9 alone.

### Enterobacteriaceae (Table 3)

The activity of the MetRS test agents and comparators was evaluated against 4 E. coli (2 wild type isolates, 1 ESBL isolate, and 1 NDM-1 isolate), and 2 K pneumoniae (1 wild type isolate and 1 KPC-2 isolate).

Based on the activity observed in M9 alone against E. coli, test compound AB12038 was the most active across isolates while AB12039 and AB12040 were also active but only against E. coli ATCC 25922 (wild type/QC isolate). AB3609 showed comparatively less activity at 24 hr and this activity was not apparent at 48 hr. AB3354 was inactive. The observed activity of the test compounds was greatly diminished upon supplementation of M9 with 1% and 5% of the standard CLSI medium.

There was no activity observed for the test compounds against K pneumoniae excluding AB12038 and AB3609 which were active only at the highest test concentration (256 μg/mL) at 24 hr against the wild type and KPC-2 isolates, respectively. Activity with the comparator agents was as expected and was consistent in M9 supplemented with 1% or 5% CLSI standard medium and M9 alone.

### Lactose Non-fermenting Gram-negative Bacilli (Table 4)

The activity of the MetRS test agents and comparators was evaluated against 2 P. aeruginosa (1 wild type isolate and 1 IMP-7/MDR isolate), and 2 A. baumannii (1 wild type isolate and 1 OXA-27/MDR isolate).

There was no activity observed with the test compounds regardless of medium with the exception of AB3609 which had activity limited to the 2 evaluated A. baumannii isolates at the highest concentrations evaluated (128 and 256 μg/mL).

Activity with the comparator agents was as expected and was consistent in M9 supplemented with 1% or 5% CLSI standard medium and M9 alone.

### H. Influenzae (Table 5)

The activity of the MetRS test agents and comparators was evaluated against 2 H. influenzae (1 wild type isolate and 1 beta-lactamase negative/ampicillin-resistant isolate). There was insufficient growth to evaluate MICs in M9 and M9 supplemented with 1% CLSI standard medium. However, there was sufficient growth at 48 hr in M9 supplemented with 5% CLSI standard medium and that data showed that AB12038 was the most potent compound, followed by AB12040. There was comparatively less activity observed with AB3609 and AB 12039 and at 48 hr AB3354 was inactive. Activity with the comparator agents was as expected where able to be determined with potent MIC values for all agents.

N. gonorrhoeae (Table 5)

The activity of the MetRS test agents and comparators were evaluated against 3 N. gonorrhoeae (1 wild type isolate, 1 cefotaxime-resistant isolate, and 1 ciprofloxacin-resistant isolate).

There was insufficient growth to evaluate MICs in M9 for 2 of 3 isolates and in M9 supplemented with 1% CLSI standard medium for 1 of 3 isolates. However, based on the available data it appeared that the supplementation of M9 media with media recommended for the growth of gonococci had little negative impact on the activity of the test compounds in contrast with other test media (MHB II). Based on the data, compound AB3609 had the most potent activity across isolates though all compounds were potent against the cefotaxime-resistant and ciprofloxacin-resistant isolates.

Activity with the comparator agents was as expected where able to be determined with potent MIC values for all agents and little evidence of altered activity for M9 supplemented with gonococcal growth medium compared to M9 alone.

C. difficile (Table 6)

The activity of the MetRS test agents and comparators was evaluated against 2 C. difficile (1 wild type isolate and 1 cytotoxin-positive isolate). There was insufficient growth to evaluate MICs in M9 and M9 supplemented with 1% CLSI standard medium. For M9 supplemented with 5% CLSI standard medium, only the wild type isolate grew enough to evaluate an MIC at 48 hr and growth was poor. Based on MICs read with poor growth, it appeared that all test compounds were active at the lowest test concentration evaluated (MICs were ≤0.25 μg/mL) for the wild type isolate at 48 hr with the exception of AB12038 which was inactive at the highest test concentration (256 μg/mL). However, because growth was poor, these MIC values should not be taken as representative data for this compound.

Activity with the comparator agents was as expected where able to be determined with potent MIC values for all agents excluding mupirocin.

### SUMMARY

In summary, varied activity was observed for the test MetRS inhibitors across the evaluated organisms. Against Gram-positive cocci, AB3609 appeared to be the most potent compound against staphylococci and enterococci, but was inactive against streptococci. AB12039 and AB12040 were active against all Gram-positive cocci though they were less potent than AB3609 for staphylococci and enterococci. Among Gram-negative bacilli, AB12038 showed potent activity against E. coli, AB3609 was the only agent with activity against A. baumannii, and there was little evidence of activity against K pneumoniae or P. aeruginosa across the evaluated test compounds. AB and AB12040 were the most potent test compounds against H. influenzae, and AB3069 was the most potent test compound against N. gonorrhoeae though all compounds showed activity against resistant gonococcal isolates. All compounds except AB12038 appeared to have potent activity against C. difficile although this assessment is based off of data from one isolate that grew poorly in the assay. AB3354 was consistently the least active test compound. Existing resistance mechanisms among the selected strains did not appear to impact the observed activity of the test compounds. Testing in M9 medium supplemented with CLSI standard media negatively impacted the activity of all test compounds, with the exception of the standard gonococcal growth medium which appeared to have little effect on activity observed with M9 alone.

### REFERENCES

1.) Clinical and Laboratory Standards Institute (CLSI). Methods for Dilution Antimicrobial Susceptibility Tests for Bacteria That Grow Aerobically; Approved Standard—Tenth Edition. CLSI document M07-A10. CLSI, 940 West Valley Road, Suite 1400, Wayne, Pa. 19087-1898 USA, 2015.

2.) CLSI. Performance Standards for Antimicrobial Susceptibility Testing; Twenty-Sixth Informational Supplement. CLSI document M100-S26. CLSI, 940 West Valley Road, Suite 1400, Wayne, Pa. 19087-1898 USA, 2016.

3.) CLSI. Methods for Antimicrobial Dilution and Disk Susceptibility Testing of Infrequently Isolated or Fastidious Bacteria; Approved Guideline-Second Edition. CLSI document M45-A2 (ISBN 1-56238-732-4). CLSI, 940 West Valley Road, Suite 1400, Wayne, Pa. 19087-1898 USA, 2010.

4.) CLSI. Methods for Antimicrobial Susceptibility Testing of Anaerobic Bacteria; Approved Standard—Seventh Edition. CLSI document M11-A7 [ISBN 1-56238-626-3]. CLSI, 940 West Valley Road, Suite 1400, Wayne, Pa. 19087-1898 USA, 2007.

5.) CLSI. Performance Standards for Antimicrobial Susceptibility Testing of Anaerobic Bacteria; Informational Supplement. CLSI document M11-S1. CLSI, 940 West Valley Road, Suite 1400, Wayne, Pa. 19087-1898 USA, 2011.

TABLE 7
MIC values¹ (μg/mL) of MetRS inhibitors and comparators against ATCC quality control organisms using
standard CLSI media

| | MMX/ | | | | | | | | | |
|---|---|---|---|---|---|---|---|---|---|---|
| | ATCC | | | | | | | | | |
| Organism | No. | AB12038 | AB12039 | AB12040 | AB3354 | AB3609 | FEP | CIP | MEM | MUP |
| Staphylococcus | 100/29213 | >256, | >256, | >256, | >256, >256 | >256, >256 | 16, 16 | 0.5, 0.5 | 0.06, 0.12 | |
| aureus | | >256 | >256 | >256 | | | (4-16) | (0.12-0.5) | (0.03-0.12) | |
| Enterococcus | 101/29212 | >256, | >256, | >256, | >256, >256 | >256, >256 | >64, >64 | 1, 1 | 4, >4 | 256, |
| faecalis | | >256 | >256 | >256 | | | | (0.25-2) | (2-8) | >256 |
| Streptococcus | 1195/49619 | >256, | >256, | >256, | >256, >256 | >256, >256 | 0.5, 0.5 | 1, 1 | 0.06, 0.06 | |
| pneumoniae | | >256 | >256 | >256 | | | | | (0.03-0.25) | |
| Escherichia | 102/25922 | 32, | >256, | >256, | >256, >256 | >256, >256 | 0.25, 0.25 | 0.008, 0.008 | 0.03, 0.03 | >256, |
| coli | | 64 | >256 | >256 | | | (0.06-0.5) | (0.004-0.015) | (0.008-0.06) | >256 |
| Pseudomonas | 103/27853 | >256, | >256, | >256, | >256, >256 | >256, >256 | 1, 2 | 0.5, 0.5 | 0.5. 1 | >256, |
| aeruginosa | | >256 | >256 | >256 | | | (1-4) | (0.25-1) | (0.25-1) | >256 |
| Haemophilus | 1224/49247 | 16, | >256, | 128, | >256, >256 | >256, >256 | 0.25, 0.25 | 0.015, 0.015 | 0.06, 0.06 | |
| influenzae | | 32 | >256 | >256 | | | (0.12-1) | (0.004-0.03) | | |
| Clostridium | 4381/700057 | ND, | ND, | ND, | | | ND, 32 | ND, >4 | ND, 1 | ND, |
| difficile | | >256 | 0.5 | 0.5 | | | | | | >256 |
| Neisseria | 683/49226 | >256, | >256, | >256, | >256, >256 | >256, >256 | 16, 32 | 0.5. 1 | >4, >4 | >256, |
| gonorrhoeae | | >256 | >256 | >256 | | | | | | >256 |

MMX = Micromyx;
ATCC = American Type Culture Collection;
FEP = cefepime;
CIP = ciprofloxacin;
MEM = meropenem;
MUP = mupirocin;
ND = not determined
¹24 hr results, 48 hr results
²CLSI QC ranges shown in parenthesis

### ADDITIONAL EMBODIMENTS AND WORKING EXAMPLES II

Efficacy of a novel test article in the murine superficial skin infection model:

### Protocol

### Purpose

Evaluation of high dose of novel test article(s) for the treatment of a superficial skin infection in mice caused by Escherichia coli.

### Methods

All studies were performed following UNTHSC approved protocols IACUC-2016-0046 which is comparable to other reported methods for these indications and has been approved by the institutional animal care and use committee (IACUC).

### Bacteria

Escherichia coli UNT116-1 (ATCC25922) was used in this model. Strain has been previously used separately for this infection at UNTHSC.

### Mice

Female 5-6 week old CD-1 (18-22 gm) were used in the studies. The mice were housed in groups of 5 with free access to food and water during the study.

### Superficial Skin Infection Model

### PreTreatment

Cytoxan 150 mg/kg@15 mg/mL on Day-4 ONLY.

### Wound

The mice were anesthetized and kept sedated during the initial procedure under isoflurane vapors (3%). The fur on the back dorsal surface was shaved using electric clippers followed by ‘wet shaving’ with a disposable razor. The skin was then sterilized with a betadine wash followed by alcohol swab. An area of the shaved skin was then abraded utilizing a sterile gauze pad. Following this procedure, the skin became visibly damaged and characterized by reddening and glistening but no bleeding. Microscopically, this procedure resulted in the controlled removal of most of the epidermal layer, with only a few basal epidermal cells remaining. The skin was then wiped with an alcohol swab and allowed to dry completely.

### Infection

After stripping of the skin, a bacterial infection was initiated by placing on the skin a 5-10 μL droplet containing approximately ˜106 cells of both bacterium concentrated from overnight cultures in stationary phase.

TABLE 1
Skin Efficacy Study Design

| | | | Con- | Dose | | | Sample | |
|---|---|---|---|---|---|---|---|---|
| Group | Compound | Formulation | centration | Regimen* | Volume | Route | time | N = |
| 1 | Test | 0.05% Methyl | 500 | +4, 8, 12, | 50 uL | Topical | 24 hr | 10 |
| | Article 1 | cellulose | | 16 & 20 hr | | | | |
| 2 | Test | | 500 | | | | | 10 |
| | Article 2 | | | | | | | |
| 3 | Gentamicin | | 0.3% | | | | | 10 |
| 4 | Infection | na | +4 hours | na | Na | na | +4 hr | 5 |
| 5 | Controls | | +24 hours | | | | Day 2 | 10 |

*time post-infection

### Timeline

Day 1: Infect & Treat starting +4 hr, 8, 12, 16, 20 hr post-infection

Day 2: Sample all Groups

### Formulations

Sponsors test articles were prepared in 0.05% methylcellulose. Fresh formulations were prepared for each day of dosing.

### Treatment

The first application of each compound (N=10 mice/group) to the stripped skin of the mice was at 4 hr post-infection then continued q4 hr. For each treatment ˜50 uL of formulation was applied. Infection control groups' untreated mice were included with each experiment. The test was terminated 4 h after the last topical treatment. Immediately after the mice were euthanized (photographs of the wounds were taken for each group), the wounds, approximately 2 cm2, were excised, rinsed in sterile PBS and homogenized together with 2 ml of phosphate-buffered saline in a Polytron tissue homogenizer. The homogenates were washed once in phosphate-buffered saline to decrease the concentration of ointment. Suitable dilutions of the homogenates were plated on appropriate agar (with the addition of charcoal to avoid the effects of antibiotic carryover) for the organism to determine the number of living bacteria (CFU).

### Data/Analysis/Reports

Draft data was supplied immediately following the study as plate counts are finalized and was in the from of an excel worksheet listing mean group CFU values with standard deviations. The final report will contain the original protocol, mean CFU values, std. dev., log reductions in treatment groups vs untreated and/or vehicle control groups, figures (bar graphs or other if applicable), appendix with individual animal data and narrative describing the results obtained.

### RESULTS

The results of the initial study are presented in Table 2. The mean bacterial skin titers indicate a 1.5-2 log CFU reduction as compared to the 24 hr untreated control group following application of the two Sponsor test articles. It was, however, noted that the wounds from these animals were incorrectly first sprayed with 70% EtOH prior to sampling. This may have affected the overall viability of the E. coil strain in the wound. Samples for the gentamicin and control animals were processed in the proper manner and the results were in agreement with those previously obtained for the model. A subsequent virulence study (data not shown) indicated a 0.6 log CFU lower mean bacterial titer from wounds processed by EtOH spray vs. the proper protocol procedure. Following consultation with the Sponsor, a repeat study was initiated with higher dose concentrations for each test article. The results of the repeat efficacy study, with AB3609 and AB12038, are presented on Table 3. Mice were infected with 6.65 log10 CFU of UNT116-1 resulting in mean bacterial skin titers of 3.82 log10 CFU/wound at 4 hours post infection and 8.44 log10 CFU/wound at 24 hours post infection for the untreated control groups. AB3609 was formulated as indicated by the sponsor, and had no issues during

application. AB12038 was formulated as indicated by the sponsor, however the test article formed a paste like substance (noted prior to the 8 hr treatment time), but was still applied to the infection site at an estimated 50 uL volume. AB3609 reduced the bacterial skin burden by 1.27 log10 CFU/wound compared to the 24 hour untreated control (7.17 mean log10 CFU/wound vs. 8.44 mean log10 CFU/wound). AB12038 reduced the bacterial skin burden by 2.95 log10 CFU/wound compared to the 24 hour untreated control (5.49 mean log10 CFU/wound vs. 8.44 mean log10 CFU/wound). Gentamicin at 0.30% reduced the bacterial skin burden by 5.98 log10 CFU/wound compared to the 24 hour untreated control (2.46 mean log10 CFU/wound vs. 8.44 mean log10 CFU/wound).

TABLE 2
Skin Efficacy Study #1

| | | | | | | Mean | | Mean | Mean |
|---|---|---|---|---|---|---|---|---|---|
| | | | | | TIME | log₁₀ | | log10 | log10 |
| | | Dose | | | (post- | CFU/ | | Change | Change vs. |
| Test Article | Concentration | Regimen | Volume | Route | infection) | wound | SD | vs. +4 hr | +24 hr |
| AB3609 | 500 mg/mL | +4, 8, 12, | 50 uL | Topical | +24 hr | 6.24 | 0.81 | 6.24 | −1.48 |
| AB12038 | 500 mg/mL | 16, & 20 | | | | 5.80 | 0.49 | 5.80 | −1.93 |
| Gentamicin | 0.30% | hr | | | | 2.35 | 0.00 | 2.35 | −5.37 |

| | | | | | | | | |
|---|---|---|---|---|---|---|---|---|
| Untreated Infection Controls | na | n | na | +4 hr | 3.33 | 0.69 | 3.33 | −4.40 |
| | na | n | na | +24 hr | 7.73 | 0.42 | 7.73 | 0.00 |

TABLE 3
Skin Efficacy Study #2

| | | | | | | Mean | | Mean | Mean |
|---|---|---|---|---|---|---|---|---|---|
| | | | | | TIME | log10 | | log10 | log10 |
| | | Dose | | | (post- | CFU/ | | Change | Change |
| Test Article | Concentration | Regimen | Volume | Route | infection) | wound | SD | vs. +4 hr | vs. +24 hr |
| AB3609 | 700 mg/mL | +4, 8, | 50 uL | Topical | +24 hr | 7.17 | 0.39 | 3.35 | −1.27 |
| AB12038 | 700 mg/mL | 12, 16, | | | | 5.49 | 0.89 | 1.67 | −2.95 |
| Gentamicin | 0.3 | & 20 hr | | | | 2.46 | 0.23 | −1.36 | −5.98 |
| Untreated Infection Controls | | na | n | na | +4 hr | 3.82 | 0.64 | 0 | −4.62 |
| | | na | n | na | +24 hr | 8.44 | 0.36 | 4.62 | 0 |

### SUMMARY

The results of this study indicate that AB3609 and AB12038 demonstrate therapeutic potential for the treatment of E. coli wound infections and warrants further investigation into dosing concentrations, formulations and regimens.

### Appendix 2. Individual Animal Data.

| | | | | | Mean | | | |
|---|---|---|---|---|---|---|---|---|
| | | | TIME | | log₁₀ | | Mean log₁₀ | Mean log₁₀ |
| | | Route, | (post- | Log ₁₀ CFU/ | CFU/ | Standard | Change | Change vs. |
| Group | Compound | Regimen | infection) | wound | wound | Deviation | vs. +4 hr | +24 hr |
| 1 | AB3609 | Topical: | +24 hr | 7.20 | 7.17 | 0.39 | 3.35 | −1.28 |
| | 700 mg/mL | +4, 8, 12, | | 7.43 | | | | |
| | | 16, & 20 | | 6.95 | | | | |
| | | hr | | 7.31 | | | | |
| | | | | 7.50 | | | | |
| | | | | 7.26 | | | | |
| | | | | 7.47 | | | | |
| | | | | 6.20 | | | | |
| | | | | 6.98 | | | | |
| | | | | 7.39 | | | | |
| 2 | AB12038 | | | 6.05 | 5.49 | 0.89 | 1.67 | −2.95 |
| | 700 mg/mL | | | 4.88 | | | | |
| | | | | 3.67 | | | | |
| | | | | 6.53 | | | | |
| | | | | 5.50 | | | | |
| | | | | 5.20 | | | | |
| | | | | 5.61 | | | | |
| | | | | 6.84 | | | | |
| | | | | 5.13 | | | | |
| | | | | 5.50 | | | | |
| 3 | Gentamicin | | | 2.83 | 2.46 | 0.23 | −1.36 | −5.98 |
| | 0.30% | | | 2.35 | | | | |
| | | | | 2.35 | | | | |
| | | | | 2.35 | | | | |
| | | | | 2.35 | | | | |
| | | | | 2.35 | | | | |
| | | | | 2.35 | | | | |
| | | | | 2.95 | | | | |
| | | | | 2.35 | | | | |
| | | | | 2.35 | | | | |
| 4 | Infection | na | +4 hr | 2.95 | 3.82 | 0.64 | 0.00 | −4.62 |
| | Controls | | | 4.05 | | | | |
| | | | | 3.35 | | | | |
| | | | | 4.43 | | | | |
| | | | | 4.31 | | | | |
| 5 | Infection | na | +24 hr | 8.50 | 8.44 | 0.36 | 4.62 | 0.00 |
| | Controls | | | 8.13 | | | | |
| | | | | 8.61 | | | | |
| | | | | 9.20 | | | | |
| | | | | 8.05 | | | | |
| | | | | 8.39 | | | | |
| | | | | 8.67 | | | | |
| | | | | 8.58 | | | | |
| | | | | 7.99 | | | | |
| | | | | 8.31 | | | | |

### Drug Carryover

Below the limit of detection

Statistical Outlier; ROUT (Q=5%) & Grubb's (Alpha=0.05)

### Appendix 3. Tukey Post Hoc Analysis: Ordinary One-Way ANOVA.

| Tukey's multiple comparisons | | Adjusted |
|---|---|---|
| test | 95.00% CI of diff. | P Value |
| AB3609 700 mg/mL vs. +24 day | −1.967 to −0.5812 | <0.0001 |
| Infection | | |
| AB12038 700 mg/mL vs. +24 day | −3.645 to −2.259 | <0.0001 |
| Infection | | |
| Gentamicin 0.30% vs. +4 hr | −2.209 to −0.5115 | 0.0004 |
| Infection Control | | |
| Gentamicin 0.30% vs. +24 day | −6.678 to −5.292 | <0.0001 |
| Infection | | |

1-14. (cancelled)

15. A compound of formula I: 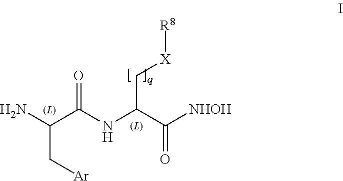
where:
R⁸ is selected from the group consisting of hydrogen and C₁-C₄ alkyl;
Ar is selected from the group consisting of phenyl, 4-hydroxyphenyl and 3-indolyl;
q is 1, 2, 3, or 4;
X is selected from the group consisting of S, O, SO, SO₂ and CH₂; and
each (L) indicates an L isomer at that stereochemical center;
including pharmaceutically acceptable salts and/or solvates thereof.

16. The compound of claim 15, wherein X is S, R⁸ is methyl, q is 1 or 2 and Ar is phenyl.

17. The compound of claim 15, wherein X is S, R⁸ is methyl, q is 1 or 2 and Ar is 4-hydroxyphenyl.

18. The compound of claim 15, wherein X is S, R⁸ is methyl, q is 1 or 2 and Ar is 3-indolyl.

19. The compound of claim 15, wherein the compound is selected from the group consisting of the L,L isomer of: 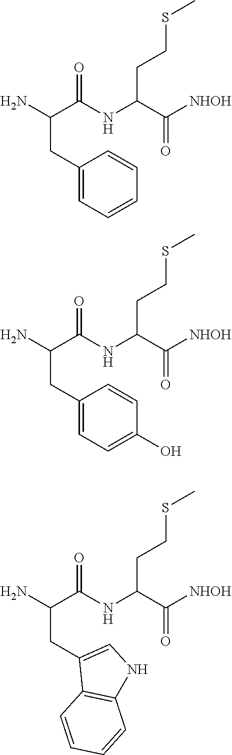
including pharmaceutically acceptable salts and/or solvates thereof.

20. A pharmaceutical composition comprising a pharmaceutically acceptable carrier and a compound of claim 15.

21. A bacteria population comprising within the intracellular space at least some of the bacteria a mixture of a compound of claim 15 or of formula II: 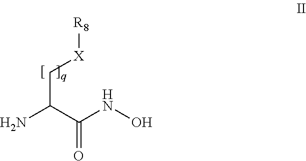
where X, q and R⁸ are as defined above; and including salts and/or hydrates thereof.

22. A method for killing prokaryotic cells which method comprises administering to said cells a compound according to claim 15.

23. The method of claim 22, wherein the prokaryotic cells are bacterial cells.

24. The method of claim 23, wherein the bacterial cells are E. coli bacteria.

25. A method of treating a subject with a bacterial infection which method comprises administering to the subject an effective amount of a compound of claim 15.

26. A method of treating a subject with a bacterial infection which method comprises administering to the subject an effective amount of a pharmaceutical composition comprising a compound of claim 15.

27. A compound of formula I: 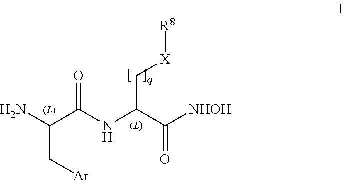
where:
R⁸ is selected from the group consisting of hydrogen and C₁-C₄ alkyl;
Ar is selected from the group consisting of phenyl, phenyl glycine, 4-hydroxyphenyl and 3-indolyl;
q is 1, 2, 3, or 4;
X is selected from the group consisting of S, O, SO, SO₂ and CH₂; and
each (L) indicates an L isomer at that stereochemical center;
including pharmaceutically acceptable salts and/or solvates thereof.

28. A pharmaceutical composition comprising a pharmaceutically acceptable carrier and a compound of claim 27.

29. A method for killing prokaryotic cells which method comprises administering to said cells a compound according to claim 27.

30. The method of claim 29, wherein the prokaryotic cells are bacterial cells.

31. The method of claim 30, wherein the bacterial cells are E. coli bacteria.

32. A method of treating a subject with a bacterial infection which method comprises administering to the subject an effective amount of a compound of claim 27.

33. A method of treating a subject with a bacterial infection which method comprises administering to the subject an effective amount of a pharmaceutical composition comprising a compound of claim 27.

34. A compound of formula I: 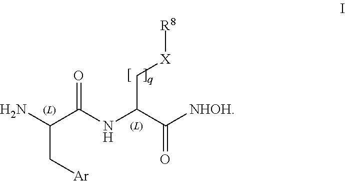
where:
R⁸ is selected from the group consisting of hydrogen and C₂-C₄ alkyl;
Ar is a histidine group;
q is 1, 2, 3, or 4;
X is selected from the group consisting of S, O, SO, SO₂ and CH₂; and
each (L) indicates an L isomer at that stereochemical center;
including pharmaceutically acceptable salts and/or solvates thereof.
